SEQUENCE LISTING (16 sequences)

SEQ ID NO: 1 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - DNA, 1600 bp, Homo sapiens
aaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 3 - DNA, 537 bp, Artificial Sequence
  codon optimized human IL-10 sequence
atgcacagctcagcactgctgtgctgcctggtcctgctgacaggggtgagggcaagcccaggccagggaacccaatctgagaacagctgcacccacttccctggcaatctgcctaacatgctgcgcgacctccgagatgccttcagcagagtgaagacttttttccagatgaaggatcagctggacaacctgctgctgaaggagtccctcctggaggactttaagggctacctgggatgccaggccctgtctgagatgatccaattctacctggaagaagttatgccccaggctgagaaccaggacccagacattaaggcccatgtcaactccctgggggaaaatctgaagaccctcaggctgcggctacggcgctgtcaccgttttctgccctgtgagaataagagcaaggctgtggagcaggtgaagaacgccttcaataagctccaggagaagggtatctacaaagcgatgagtgaatttgatatcttcattaattatatagaagcttatatgacaatgaaaatcagaaactga

SEQ ID NO: 4 - protein, 185 aa, Homo sapiens
MKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 5 - DNA, 558 bp, Homo sapiens
atgaagttatgggatgtcgtggctgtctgcctggtgctgctccacaccgcgtccgccttcccgctgcccgccgcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 6 - DNA, 558 bp, Artificial Sequence
  Codon optimized human GDNF DNA
atgaaactttgggacgtggtggctgtctgcctggtgctcctccacaccgccagtgcgtttccgctgcccgccgctaacatgccagaggattatcctgatcagttcgatgatgttatggacttcattcaagccacaatcaagcggctgaaacgatcaccagataaacagatggcagtgcttcctcgccgcgagcgtaatcggcaggctgcagcagccaatcccgagaattcccgaggaaaagggcgcaggggtcagaggggcaagaaccgggggtgtgtcctgactgcaatacatttaaacgtgactgacttgggtctgggctatgagaccaaggaagaactcattttcaggtactgcagcggctcttgcgatgccgcggaaacaacgtacgacaaaatcttgaagaacctctccagaaacagaaggctggtgagtgacaaggtaggacaggcctgttgcagacccatcgcctttgacgacgatctgagctttctggatgacaatctggtttaccacatcctacggaagcattctgctaaaagatgtggatgtatttga

SEQ ID NO: 7 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 8 - DNA, 2824 bp, Homo sapiens
gcggccgcccgcacttcccgcctctggctcgcccgaggacgcgctggcacgcctcccaccccctcactctgactccagctggcgtgcatggtctgcctcgcatcctcacgactcagctccctccctctctcgtgtttttttcctccgccgccccctcattcatccccactgggctccctttccctcaaatgctctggggctctccgcgctttcctgagtccgggctccgaggacccttaggtagtcccggtctcttttaaagctccccggcttccaaagggttgccacgtccctaaaccctgtctccagctcgcatacacacacgcacagacacgcacgttttctgttcctgcgtgacacccgccctcgccgctcggccccgccggtccccgcgcggtgccctcctcccgccacacgggcacgcacgcgcgcgcagggccaagcccgaggcagctcgcccgcagctcgcactcgcaggcgacctgctccagtctccaaagccgatggcatctccgggctctggcttttggtctttcgggtcggaagatggctctggggattccgagaatcccggcacagcgcgagcctggtgccaagtggctcagaagttcacgggcggcatcggaaacaaactgtgcgccctgctctacggagacgccgagaagccggcggagagcggcgggagccaacccccgcgggccgccgcccggaaggccgcctgcgcctgcgaccagaagccctgcagctgctccaaagtggatgtcaactacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacacaagatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagatttataataaccttgctcaccaagctgttccacttctctagagaacatgccctcagctaagccccctactgagaaacttcctttgagaattgtgcgacttcacaaaatgcaaggtgaacaccactttgtctctgagaacagacgttaccaattatggagtgtcaccagctgccaaaatcgtaggtgttggctctgctggtcactggagtagttgctactcttcagaatatggacaaagaaggcacaggtgtaaatatagtagcaggatgaggaacctcaaactgggtatcattttgcacgtgctcttctgttctcaaatgctaaatgcaaacactgtgtatttattagttaggtgtgccaaactaccgttcccaaattggtgtttctgaatgacatcaacattcccccaacattactccattactaaagacagaaaaaaataaaaacataaaatatacaaacatgtggcaacctgttcttcctaccaaatataaacttgtgtatgatccaagtattttatctgtgttgtctctctaaacccaaataaatgtgtaaatgtggacacatctc

SEQ ID NO: 9 - protein, 594 aa, Homo sapiens
MASSTPSSSATSSNAGADPNTTNLRPTTYDTWCGVAHGCTRKLGLKICGFLQRTNSLEEKSRLVSAFKERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMFTYEIAPVFVLMEQITLKKMREIVGWSSKDGDGIFSPGGAISNMYSIMAARYKYFPEVKTKGMAAVPKLVLFTSEQSHYSIKKAGAALGFGTDNVILIKCNERGKIIPADFEAKILEAKQKGYVPFYVNATAGTTVYGAFDPIQEIADICEKYNLWLHVDAAWGGGLLMSRKHRHKLNGIERANSVTWNPHKMMGVLLQCSAILVKEKGILQGCNQMCAGYLFQPDKQYDVSYDTGDKAIQCGRHVDIFKFWLMWKAKGTVGFENQINKCLELAEYLYAKIKNREEFEMVFNGEPEHTNVCFWYIPQSLRGVPDSPQRREKLHKVAPKIKALMMESGTTMVGYQPQGDKANFFRMVISNPAATQSDIDFLIEEIERLGQDL

SEQ ID NO: 10 - DNA, 1784 bp, Homo sapiens
atggcgtctcgaccccatcttcgtccgcaacctcctcgaacgcgggagcggaccccaataccactaacctgcgccccacaacgtacgatacctggtgcggcgtggcccatggatgcaccagaaaactggggctcaagatctgcggcttcttgcaaaggaccaacagcctggaagagaagagtcgccttgtgagtgccttcaaggagaggcaatcctccaagaacctgctttcctgtgaaaacagcgaccgggatgcccgcttccggcgcacagagactgacttctctaatctgtttgctagagatctgcttccggctaagaacggtgaggagcaaaccgtgcaattcctcctggaagtggtggacatactcctcaactatgtccgcaagacatttgatcgctccaccaaggtgctggactttcatcacccacaccagttgctggaaggcatggagggcttcaacttggagctctctgaccaccccgagtccctggagcagatcctggttgactgcagagacaccttgaagtatggggttcgcacaggtcatcctcgatttttcaaccagctctccactggattggatattattggcctagctggagaatggctgacatcaacggccaataccaacatgtttacatatgaaattgcaccagtgtttgtcctcatggaacaaataacacttaagaagatgagagagatagttggatggtcaagtaaagatggtgatgggatattttctcctgggggcgccatatccaacatgtacagcatcatggctgctcgctacaagtacttcccggaagttaagacaaagggcatggcggctgtgcctaaactggtcctcttcacctcagaacagagtcactattccataaagaaagctggggctgcacttggctttggaactgacaatgtgattttgataaagtgcaatgaaagggggaaaataattccagctgattttgaggcaaaaattcttgaagccaaacagaagggatatgttcccttttatgtcaatgcaactgctggcacgactgtttatggagcttttgatccgatacaagagattgcagatatatgtgagaaatataacctttggttgcatgtcgatgctgcctggggaggtgggctgctcatgtccaggaagcaccgccataaactcaacggcatagaaagggccaactcagtcacctggaaccctcacaagatgatgggcgtgctgttgcagtgctctgccattctcgtcaaggaaaagggtatactccaaggatgcaaccagatgtgtgcaggatacctcttccagccagacaagcagtatgatgtctcctacgacaccggggacaaggcaattcagtgtggccgccacgtggatatcttcaagttctggctgatgtggaaagcaaagggcacagtgggatttgaaaaccagatcaacaaatgcctggaactggctgaatacctctatgccaagattaaaaacagagaagaatttgagatggttttcaatggcgagcctgagcacacaaacgtctgtttttggtatattccacaaagcctcaggggtgtgccagacagccctcaacgacgggaaaagctacacaaggtggctccaaaaatcaaagccctgatgatggagtcaggtacgaccatggttggctaccagccccaaggggacaaggccaacttcttccggatggtcatctccaacccagccgctacccagtctgacattgacttcctcattgaggagatagaaagactgggccaggatctgtaa

SEQ ID NO: 11 - DNA, 28 bp, Artificial Sequence
  Forward primer for amplifying CMV promoter (F-JDK)
ttcggccgtcgaggagcttggcccattg

SEQ ID NO: 12 - DNA, 36 bp, Artificial Sequence
  Reverse primer for amplifying CMV promoter (R-JDK)
gacgtcgacctagctagcgaattcggggccgcggag

SEQ ID NO: 13 - DNA, 34 bp, Artificial Sequence
  Forward primer for amplifying SV40pA promoter (F-SV40pA)
ccatcgatcagacatgataagatacattgatgag

SEQ ID NO: 14 - DNA, 43 bp, Artificial Sequence
  Reverse primer for amplifying SV40pA promoter (R-SV40pA)
gacgtcgacgcggccgctaccacatttgtagaggttttacttg

SEQ ID NO: 15 - DNA, 43 bp, Artificial Sequence
  Forward primer for SV40-CMV-sCAG-bGHpA-Infu-F
cctgcggccggtcgactaccacatttgtagaggttttacttgc

SEQ ID NO: 16 - DNA, 35 bp, Artificial Sequence
  Reverse primer for SV40-CMV-sCAG-bGHpA-Infu-R
aataatcaatgtcgactcgaggagcttggcccatt

(19) United States
(12) Patent Application Publication
(10) Pub. No.: US 2025/0276041 A1
(43) Pub. Date: Sep. 4, 2025

(54) COMPOSITION FOR PREVENTING OR TREATING DEGENERATIVE BRAIN DISEASES

(71) Applicant: KOLON LIFE SCIENCE, INC., Seoul (KR)

(72) Inventors: Jangjoon PARK, Seoul (KR); Jaeil SHIN, Seoul (KR); Soonoh HONG, Seoul (KR); Kyungran KIM, Gyeonggi-do (KR); Minju KIM, Seoul (KR); Hyelin JI, Seoul (KR); Yeomoon SIM, Seoul (KR); Haeun KIM, Seoul (KR); Juyoun LEE, Seoul (KR); Hyejin LEE, Incheon (KR)

(73) Assignee: KOLON LIFE SCIENCE, INC., Seoul (KR)

(21) Appl. No.: 18/281,412

(22) PCT Filed: Mar. 11, 2022
(86) PCT No.: PCT/KR2022/003444

§ 371 Date: Sep. 11, 2023

(30) Foreign Application Priority Data
Mar. 12, 2021 (KR) 10-2021-0032759

(51) Int. Cl. A61K 38/20; A61K 38/18; A61K 38/51; A61K 48/00; A61P 25/28

(52) U.S. Cl. CPC A61K 38/2066; A61K 38/185; A61K 38/51; A61K 48/005; A61P 25/28; C12Y 401/01015

(57) ABSTRACT

The present invention relates to a composition for preventing or treating neurodegenerative diseases. The pharmaceutical composition of the present invention contains at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same. Since the pharmaceutical composition of the present invention exhibits a better effect of alleviating and preventing neurodegenerative diseases by co-administration of active ingredients than upon single administration, the pharmaceutical composition of the present invention can exhibit the same or higher effect only with a low concentration of the active ingredients. Therefore, the pharmaceutical composition of the present invention can be advantageously used for the prevention or treatment of neurodegenerative diseases.

### TECHNICAL FIELD

The present invention relates to a composition for preventing or treating neurodegenerative diseases and a method for preventing or treating neurodegenerative diseases using the same.

### BACKGROUND ART

A neurodegenerative disease is currently one of the most socially problematic diseases, with a growing elderly population. However, there is no effective therapeutic agent (=drugs) to treat this disease, thus peoples are paying attention to develop prevention and treatment of neurodegenerative disease. A neurodegenerative disease is a general term for diseases caused by the death of neurons, which is progressing slowly and steadily in one or more parts of the nervous system, and a neurodegenerative disease may occur when the brain is damaged by the structural deterioration of neuronal cell in the brain due to aging, secondary symptoms caused by adult diseases such as circulatory disorders, or physical and mechanical factors such as traffic accidents, industrial accidents, and carbon monoxide poisoning, or even when brain tissue is irreversibly damaged by reduced or blocked blood flow to the brain, but the exact cause of disease is currently unknown. When a neurodegenerative disease occurs, progressive cognitive dysfunction, progressive ataxia, muscle weakness, amyotrophy, and the like symptoms may occur depending on the location of the lesion in the brain and pathogenesis of the disease, and the like. Each of various genes is known to be effective in suppressing neuronal cell death. However, single gene therapy has a limitation in that it cannot sufficiently suppress the death of neuronal cells, and since a high dose with single gene therapy is injected to achieve the therapeutic effect, it is not free from side effects such as toxicity. Therefore, there is need for developing a therapy not only effectively preventing or treating neurodegenerative diseases without side effects, but also exhibiting a synergistic effect on the preventive or therapeutic effects of neurodegenerative diseases according to the co-administration of therapeutic genes.

### DISCLOSURE

### Technical Problem

Under the background as described above, the present inventors conducted intensive studies to develop a therapy having preventive or therapeutic effects against neurodegenerative diseases, and as a result, confirmed that when at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same are used, the case shows a remarkably excellent preventive or therapeutic effect on neurodegenerative diseases compared to a use of only one, thereby completing the present invention.

Thus, an object of the present invention is to provide a pharmaceutical composition for preventing or treating a neurodegenerative diseases, containing at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same.

Further, another object of the present invention is to provide a health functional food for preventing or ameliorating a neurodegenerative disease, containing at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same.

In addition, still another object of the present invention is to provide a method for preventing or treating a neurodegenerative disease, the method including administering at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same to an individual in need thereof.

Furthermore, yet another object of the present invention is to provide a use of at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same for preparing a preventive or therapeutic pharmaceutical agent for neurodegenerative diseases.

However, technical problems to be solved by the present invention are not limited to the aforementioned problems, and other problems that are not mentioned may be clearly understood by those skilled in the art from the following description.

### Technical Solution

To achieve the objects, the present invention provides a pharmaceutical composition for preventing or treating a neurodegenerative disease, containing at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same.

As an exemplary embodiment of the present invention, the pharmaceutical composition may contain the IL-10 protein or the gene encoding the same and the GDNF protein or the gene encoding the same.

As another exemplary embodiment of the present invention, the pharmaceutical composition may further contain the GAD protein or the gene encoding the same.

As still another exemplary embodiment of the present invention, the gene may be contained in a vector.

As yet another exemplary embodiment of the present invention, the vector may be a viral or non-viral vector.

As yet another exemplary embodiment of the present invention, the viral vector may be at least one selected from the group consisting of adenoviruses, adeno-associated viruses (AAV), herpes simplex virus, lentiviruses, retroviruses, cytomegalovirus, baculoviruses, and poxviruses.

As yet another exemplary embodiment of the present invention, the non-viral vector may be at least one selected from the group consisting of plasmids, liposomes, cationic polymers, micelles, emulsions, and solid lipid nanoparticles.

As yet another exemplary embodiment of the present invention, the IL-10 protein may be an amino acid sequence represented by SEQ ID NO: 1.

As yet another exemplary embodiment of the present invention, the IL-10 encoding gene may be a base sequence represented by SEQ ID NO: 2 or 3.

As yet another exemplary embodiment of the present invention, the GDNF protein may be an amino acid sequence represented by SEQ ID NO: 4.

As yet another exemplary embodiment of the present invention, the GDNF protein encoding gene may be a base sequence represented by SEQ ID NO: 5 or 6.

As yet another exemplary embodiment of the present invention, the GAD may be GAD 65 or GAD 67.

As yet another exemplary embodiment of the present invention, the GAD protein may be an amino acid sequence represented by SEQ ID NO: 7 or 9.

As yet another exemplary embodiment of the present invention, the GAD encoding gene may be a base sequence represented by SEQ ID NO: 8 or 10.

As yet another exemplary embodiment of the present invention, the neurodegenerative diseases may be at least one selected from the group consisting of Parkinson's disease, dementia, Alzheimer's disease, Huntington's disease, frontotemporal lobar degeneration, dementia with Lewy bodies, vascular dementia, corticobasal degeneration, multiple system atrophy, progressive supranuclear palsy, primary lateral sclerosis, spinal muscular atrophy, stroke, cerebral infarction, head trauma, spinal cord injury, cerebral arteriosclerosis, Lou Gehrig's disease, and multiple sclerosis disease.

As yet another exemplary embodiment of the present invention, the pharmaceutical composition may additionally contain a physiologically acceptable carrier.

As yet another exemplary embodiment of the present invention, the pharmaceutical composition may be an injection.

As yet another exemplary embodiment of the present invention, the pharmaceutical composition may have an effect of ameliorating motor abnormalities caused by neuronal cell death.

As yet another exemplary embodiment of the present invention, the neurons may be motor neurons, interneurons or sensory neurons.

As yet another exemplary embodiment of the present invention, the pharmaceutical composition may have an effect of improving cognitive ability and memory.

Further, the present invention provides a health functional food for preventing or ameliorating neurodegenerative diseases, containing at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same.

In addition, the present invention provides a method for preventing or treating neurodegenerative diseases, the method including administering at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same to an individual in need thereof.

Furthermore, the present invention provides a use of at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same for preparing a preventive or therapeutic pharmaceutical agent for neurodegenerative diseases.

### Advantageous Effects

The composition of the present invention contains at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same. Therefore, since the pharmaceutical composition of the present invention exhibits a better effect of alleviating and preventing neurodegenerative diseases by co-administration of active ingredients than upon single administration, the pharmaceutical composition of the present invention can exhibit the same or higher effect only with a low concentration of the active ingredients. Thus, the pharmaceutical composition of the present invention can be advantageously used for the prevention or treatment of neurodegenerative diseases.

However, the effects of the present invention are not limited to the aforementioned effects and should be understood to include all possible effects deduced from the configuration of the invention described in the detailed description or the claims of the present invention.

### DESCRIPTION OF DRAWINGS

FIG. 1 is a result confirming the effect of co-administration of hIL-10 and hGDNF on ameliorating neurodegenerative diseases, FIGS. 1A and 1B are results confirming that, in a 6-OHDA induced neurodegenerative animal models pretreated with test articles, rAAV5-hIL-10/hGDNF administration (co-administration) is more effective in protecting dopaminergic nerve fiber than the administration of rAAV5-hIL-10 and rAAV5-hGDNF alone in the striatum region of the brain, FIG. 1B is a result confirming that, in a 6-OHDA induced neurodegenerative animal models pretreated with test articles, rAAV5-hIL-10/hGDNF administration (co-administration) is more effective in protecting dopaminergic neurons than the administration of rAAV5-hIL-10 and rAAV5-hGDNF alone in the substantia nigra pars compacta (SNpc) region, and FIG. 1C is a result confirming, through a rotation test, that rAAV5-hIL-10/hGDNF administration (co-administration) is more effective in ameliorating hypokinesia symptoms than the administration of rAAV5-hIL-10 and rAAV5-hGDNF alone.

FIG. 2 is a result confirming the long-term effect of ameliorating motor function in a neurodegenerative disease animal model. rAAV5-hIL-10/hGDNF was injected into 6-OHDA induced neurodegenerative disease model and its efficacy in improving motor function was confirmed using a rotation test.

FIG. 3 is a result confirming the neuroprotective effect of the gene therapy of the present invention on dopaminergic neurons and fibers. One of the gene therapies of the present invention (rAAV5-hIL-10/hGDNF and rAAV5-hGAD65) was pretreated and neurodegenerative disease animal model was induced by 6-OHDA to confirm the efficacy of dopaminergic neuroprotection. Specifically, FIG. 3A shows the result confirmed in the striatum, and FIG. 3B shows the result confirmed in the substantia nigra pars compacta (SNpc).

FIG. 4 showed the hypokinesia prevention effect of the gene therapy of the present invention in 6-OHDA induced neurodegenerative mouse model by pretreating one of the present invention (rAAV5-hIL-10/hGDNF and rAAV5-hGAD65). Specifically, FIGS. 4a and 4b is results of rotation test after inducing animal disease model at 4 and 10 weeks, respectively. FIG. 4c showed the results of Rota-rod test, 2 weeks post inducing animal disease model.

FIG. 5 is a set of results confirming the anti-apoptotic effect on the oxidative stress in co-administration of hIL-10 and hGDNF supernatant or simultaneous administration of hIL-10, hGDNF, and hGAD65 supernatant. The combination supernatant was produced by each therapeutic gene plasmid transfection. Specifically, FIG. 5A and FIG. 5B are confirming that the effect of inhibiting neuronal cell death is exhibited in co-administration of hIL-10 and hGDNF supernatant or simultaneous administration of hIL-10, hGDNF, and hGAD65 supernatant in the case of neuronal cell death by H₂O₂ and 6-OHDA, respectively.

### MODES OF THE INVENTION

The present inventors conducted intensive studies to develop a therapeutic pharmaceutical t having preventive or therapeutic effects against neurodegenerative diseases, and as a result, confirmed that when at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same are used, the case shows an excellent preventive or therapeutic effect on neurodegenerative diseases compared to a use of only one, thereby completing the present invention.

Thus, the present invention provides a pharmaceutical composition for preventing or treating neurodegenerative diseases, containing at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same.

As used herein, the term “prevention” refers to all actions that suppress or delay the onset of a neurodegenerative disease by administering the pharmaceutical composition provided by the present invention to an individual expected to develop the neurodegenerative disease.

As used herein, the term “treatment” refers to all actions that clinically intervene to alter the natural processes of the individual or cells to be treated, and may be carried out either during the development of a clinical pathological condition or to prevent the clinical pathological condition. The desired therapeutic effect includes preventing the occurrence or recurrence of a disease, alleviating symptoms, reducing all direct or indirect pathological consequences of the disease, preventing metastasis, reducing a disease progression rate, mitigating or temporarily alleviating a condition, making a patient feel better, or improving the prognosis. For the purpose of the present invention, the treatment may be interpreted as including all actions of administering the pharmaceutical composition of the present invention to a patient who has developed a neurodegenerative disease to ameliorate the symptoms of the neurodegenerative disease, but is not limited thereto.

A combination of the two or more may be interleukin 10 (IL-10) and glial cell line-derived neurotrophic factor (GDNF); interleukin 10 (IL-10) and glutamate decarboxylase (GAD); glial cell line-derived neurotrophic factor (GDNF) and glutamate decarboxylase (GAD); interleukin 10 (IL-10), glial cell line-derived neurotrophic factor (GDNF) and glutamate decarboxylase (GAD), and the IL-10, GDNF and GAD may be each a protein or gene encoding the same.

Further, the gene may be DNA or RNA (specifically mRNA), and may be in the form of being operably contained in a vector. Specifically, the gene may be in the form of being operably included in a viral or a non-viral vector.

The gene encoding IL-10 may be operably included in a first vector, the gene encoding GDNF may be operably included in a second vector, the gene encoding GAD may be operably included in a third vector, and two or more genes may be included in one vector.

The two or more genes selected from the group consisting of the gene encoding IL-10, the gene encoding GDNF, and the gene encoding GAD may be in the form of being included in a vector. In this case, the vector may be a viral vector, or a non-viral vector such as a plasmid or a liposome. In addition, some of the genes may be in the form of being included in a viral vector and the remaining genes may be included in the form of being included in a non-viral vector.

As an exemplary embodiment, IL-10 may be in the form of being included in a viral vector, and GDNF may be in the form of being included in a non-viral vector. Furthermore, GDNF may be in the form of being included in a viral vector, and IL-10 may be in the form of being included in a non-viral vector. Moreover, IL-10 may be in the form of being included in a viral vector, and GAD may be in the form of being included in a non-viral vector. Further, IL-10 may be in the form of being included in a viral vector, and GDNF and GAD may be in the form of being included in a non-viral vector. In addition, IL-10 and GDNF may be in the form of being included in a viral vector, and GAD may be in the form of being included in a non-viral vector. Furthermore, IL-10 and GAD may be in the form of being included in a viral vector, and GDNF may be in the form of being included in a non-viral vector. Further, IL-10 may be in the form of being included in a viral vector, and GDNF and GAD may be in the form of being included in a non-viral vector. In addition, GDNF and GAD may be in the form of being included in a viral vector, and IL-10 may be in the form of being included in a non-viral vector. Furthermore, GDNF may be in the form of being included in a viral vector, and IL-10 and GAD may be in the form of being included in a non-viral vector. Further, all of IL-10, GDNF and GAD may be in the form of being included in a viral vector. All of IL-10, GDNF and GAD may be in the form of being included in a non-viral vector.

The viral vector may be at least one selected from the group consisting of adenoviruses, adeno-associated viruses (AAV), herpes simplex virus, lentiviruses, retroviruses, cytomegalovirus, baculoviruses, poxviruses, and the like, but is not limited thereto. Preferably, the viral vector may be an adeno-associated virus.

In addition, the non-viral vector may be at least one selected from the group consisting of plasmids, liposomes, cationic polymers, micelles, emulsions, and solid lipid nanoparticles.

As used herein, the term “plasmid” refers to a circular DNA fragment that is present separately from the bacterial chromosome. The plasmid does not include genes that are essential for bacterial survival, but it includes genes that are essential for resistance to certain antibiotics and gene exchange between bacteria. Furthermore, the plasmid can proliferate independently of chromosomes, and may include a selectable marker.

As used herein, the term “liposome” refers to a vesicle formed while forming a bilayer due to a hydrophilic part and a hydrophobic part when molecules having both the hydrophobic part and the hydrophilic part, such as phospholipids, are suspended in an aqueous solution. Liposomes are separated from the outer membrane by a membrane composed of a lipid bilayer, and liposomes incorporating DNA, mRNA, and the like may be used as carriers of genetic information.

As used herein, the term “cationic polymer” refers to a material that forms a complex with anionic DNA through ionic bonding as a cationic lipid or polymer compound, and is delivered into cells.

As used herein, the term “micelle” refers to a thermodynamically stable colloidal aggregate made by the association of molecules consisting of polar groups and non-polar groups, such as surfactants and lipid molecules, by van der Waals forces in a solution. Further, micelles containing DNA, RNA, and the like may be used as carriers of genetic information.

As used herein, the term “emulsion” means that, when two solutions having different phases are mixed, one liquid forms fine particles and is dispersed in the other liquid. DNA, mRNA and the like may be included in the core of the emulsion particles and used as carriers of genetic information.

As used herein, the term “solid lipid nanoparticles” refers to a formulation in which a drug is incorporated in nano-sized microparticles consisting of a solid lipid instead of a liquid lipid.

A mixing ratio of Vector 1 (for example, a first vector) including any one gene selected from the group consisting of IL-10, GDNF and GAD and Vector 2 (for example, a second vector) including at least one gene selected from the group consisting of the remaining genes, which are not included in Vector 1, may be 1:1 to 100 or 1 to 100:1. Specifically, although not limited thereto, the mixing ratio of Vector 1 and Vector 2 may be 1:1 to 100 or 1 to 100:1 based on VG.

Vector 1 (for example, a first vector) including the gene encoding IL-10, Vector 2 (for example, a second vector) including the gene encoding GDNF, and Vector 3 (for example, a third vector) including the gene encoding GAD may be mixed in various ratios, and for example, the mixing ratio may be 1:1:1 to 1:1:100; 1:1:1 to 1:100:1; 1:1:1 to 100:1:1; 1:1:1 to 1:100:100; 1:1:1 to 100:100:1; or 1:1:1 to 100:1:100, but is not limited thereto.

As used herein, the term “operably” refers to being linked to a regulatory sequence in a manner that allows a transgene to be expressed in a host cell. The regulatory sequence is a DNA sequence that regulates gene expression, and may include promoters and other regulatory elements such as enhancers and polyadenylation. In addition, the regulatory sequence provides a binding site for the transcription factor that regulates the expression of the transgene, and may affect a complex structure with the transcription factor to determine the function of the transcription factor.

As used herein, the term “IL-10” refers to an anti-inflammatory cytokine belonging to class II cytokines (Renauld, Nat Rev Immunol, 2003). IL-10 is in the form of a homodimer consisting of two subunits, each with a length of 178 amino acids, and is also known as a human cytokine synthesis inhibitory factor (CSIF). IL-10 performs the function of suppressing the activity of natural killer (NK) cells in immune responses and forms a complex with the IL-10 receptor to participate in signal transduction. IL-10 may be a human-, mouse-, rat-, dog-, cat-, or horse-derived protein or a base sequence encoding the same, but is not limited thereto. IL-10 may consist of an amino acid sequence of SEQ ID NO: 1 of NCBI Accession No. NP_000563.1, and the gene encoding the same may be a base sequence represented by SEQ ID NO: 2 or SEQ ID NO: 3.

The IL-10 encoding gene may be a base sequence encoding an IL-10 variant that maintains IL-10 activity. The base sequence encoding the IL-10 variant may be a base sequence encoding an amino acid sequence having a sequence homology of at least 60% or more, 70% or more, 80% or more, or 90% or more with the IL-10 amino acid sequence indicated above, and may be most preferably a base sequence encoding an amino acid sequence having a sequence homology of 95% or more.

Furthermore, the base sequence encoding the IL-10 variant may be a base sequence having a sequence homology of at least 60% or more, 70% or more, 80% or more, or 90% or more with the IL-10 base sequence indicated above, and may be most preferably a base sequence having a sequence homology of 95% or more.

The “% sequence homology” is confirmed by comparing comparison regions with two sequences optimally aligned, and a portion of the base sequence in the comparison region may include an addition or deletion (that is, a gap) compared to the reference sequence (without addition or deletion) for the optimal alignment of the two sequences.

As used herein, the term “GDNF” refers to one of nerve growth factor that constitutes the GDNF ligand family, and the GDNF ligand family includes GDNF, neuturin (NRTN), artemin (ARTN), and persephin (PSPN). Further, GDNF is a protein that promotes the survival of many types of neurons, and transmits signals through GDNF receptor alpha-1 (GFR α1), GDNF receptor alpha-2 (GFR α2) and GDNF receptor alpha-3 (GFR α3). GDNF may be a human-, mouse-, rat-, dog-, cat-, or horse-derived protein or a base sequence encoding the same, but is not limited thereto. GDNF may consist of an amino acid sequence of SEQ ID NO: 4 of NCBI Accession No. NCBI NP_954701.1, and the gene encoding the same may be a base sequence represented by SEQ ID NO: 5 or SEQ ID NO: 6.

The GDNF encoding gene may be a base sequence encoding a GDNF variant that maintains GDNF activity. The base sequence encoding the GDNF variant may be a base sequence encoding an amino acid sequence having a sequence homology of at least 60% or more, 70% or more, 80% or more, or 90% or more with the GDNF amino acid sequence indicated above, and may be most preferably a base sequence encoding an amino acid sequence having a sequence homology of 95% or more.

Further, the base sequence encoding the GDNF variant may be a base sequence having a sequence homology of at least 60% or more, 70% or more, 80% or more, or 90% or more with the GDNF base sequence indicated above, and may be most preferably a base sequence having a sequence homology of 95% or more.

As used herein, the term “GAD” refers to an enzyme that decarboxylates glutamic acid to produce gamma-aminobutyric acid (GABA). The GAD may be GAD65 or GAD67. Specifically, the GAD65 may be a human-, mouse-, rat-, dog-, cat-, or horse-derived protein or a base sequence encoding the same, but is not limited thereto. The GAD65 may consist of an amino acid sequence of SEQ ID NO: 7 of NCBI Accession No. NP_000809.1, and the gene encoding the same may be a base sequence represented by SEQ ID NO: 8. The GAD67 may be a human-, mouse-, rat-, dog-, cat-, or horse-derived protein or a base sequence encoding the same, but is not limited thereto. The GAD67 may consist of an amino acid sequence of SEQ ID NO: 9 of NCBI Accession No. NP_000808.2, and the gene encoding the same may be a base sequence represented by SEQ ID NO: 10.

In addition, the GAD encoding gene may be a base sequence that encodes a GAD variant that enables GABA to be produced by maintaining GAD activity. The GAD variant includes all sequences in which the characteristics of GAD producing GABA are maintained, and is not limited to thereto, but preferably, the base sequence encoding the GAD variant may be a base sequence encoding an amino acid sequence having a sequence homology of at least 60% or more, 70% or more, 80% or more, or 90% or more with the GAD amino acid sequence indicated above, and may be most preferably a base sequence encoding an amino acid sequence having a sequence homology of 95% or more.

In addition, the base sequence encoding the GAD variant may be a base sequence having a sequence homology of at least 60% or more, 70% or more, 80% or more, or 90% or more with the GAD base sequence indicated above, and may be most preferably a base sequence having a sequence homology of 95% or more.

The pharmaceutical composition of the present invention may exhibit a preventive effect against neurodegenerative diseases through a therapeutic genes or vectors containing the genes. The composition of the present invention may achieve a better neurodegenerative disease preventive or therapeutic effect than single administration by co-administering therapeutic pharmaceutical agents having different mechanisms.

In particular, according to the present invention, when two or more genes selected from the group consisting of genes encoding IL-10, GDNF and GAD65 are co-administered, a synergistic preventive or therapeutic effect on neurodegenerative diseases shown. Therefore, the pharmaceutical composition of the present invention may be advantageously used for the prevention or treatment of neurodegenerative diseases, and specifically may ameliorate motor abnormalities in neurodegenerative diseases caused by the death of neurons, and may ameliorate cognitive and memory decline, which are the main symptoms of neurodegenerative diseases.

In the present invention, each of the vectors including IL-10, GDNF and GAD65 may be administered alone or two or more thereof may be administered together, and may also be administered in a form including two or more genes in one vector. As an administration site, IL-10 may be administered to the striatum (ST), GDNF may be administered to the striatum (ST), and GAD65 may be administered to the subthalamic nucleus (STN), but the administration is not limited thereto. The administration site of rAAV5-hGDNF/hIL-10, which is one of the aforementioned examples, may be the striatum (ST), but is not limited thereto.

In the present invention, the neurons may be motor neurons that transmit signal to the brain or spinal cord and transmit the signal to effectors such as muscle cells or secretory glands, or interneurons that are distributed throughout the central nervous system and transmit signal from sensory neurons to motor neurons, or sensory neurons that transmit signal to the brain or spinal cord to transmit the signal to effectors such as muscle cells or secretory glands.

In the present invention, the neurodegenerative disease is not limited to, but may be at least one selected from the group consisting of Parkinson's disease, dementia, Alzheimer's disease, Huntington's disease, frontotemporal lobar degeneration, dementia with Lewy bodies, vascular dementia, corticobasal degeneration, multiple system atrophy, progressive supranuclear palsy, primary lateral sclerosis, spinal muscular atrophy, stroke, cerebral infarction, head trauma, spinal cord injury, cerebral arteriosclerosis, Lou Gehrig's disease, and multiple sclerosis disease, and may be preferably Parkinson's disease, dementia or Alzheimer's disease.

The present inventors confirmed, through specific exemplary embodiments, that the pharmaceutical composition of the present invention can be advantageously used for the prevention or treatment of neurodegenerative diseases.

In an exemplary embodiment of the present invention, it was confirmed that, when rAAV5-hIL-10/hGDNF was administered to a mouse model in which a neurodegenerative disease was induced, motor dysfunction, which is a symptom induced by the neurodegenerative disease, was statistically significant ameliorated (see Example 1).

In another exemplary embodiment of the present invention, it is confirmed that pre-treatment of rAAV5-hIL-10/hGDNF and rAAV5-hGAD65 on 6-OHDA induced neurodegenerative disease model has been found to ameliorate dopaminergic neuronal cell death and motor dysfunction, which are symptoms of neurodegenerative disease. Improvement of these neurodegenerative symptoms is induced by administration of the pharmaceutical composition of the present invention. (see Example 2).

In still another embodiment of the present invention, it was confirmed that in the case of pretreatment with hIL-10 and hGDNF supernatants produced by each gene plasmid or hIL-10, hGDNF and hGAD65 supernatants produced by each gene plasmid, neuronal cell death induced by H₂O₂ or 6-OHDA was suppressed (see Example 3).

Through the aforementioned results, the present inventors were able to confirm that neurodegenerative diseases can be prevented or treated by the pharmaceutical composition of the present invention.

The pharmaceutical composition of the present invention may further include a suitable carrier, excipient, and diluent, which are typically used to prepare a pharmaceutical composition. A composition of pharmaceutically acceptable carrier can be in various forms, such as oral or parenteral formation. When the composition is formulated, the composition may be produced using a commonly used diluent or excipient, such as a filler, an extender, a binder, a wetting agent, a disintegrant, and a surfactant. A solid formulation for oral administration may include a tablet, a pill, a powder, a granule, a capsule, and the like, and the solid formulation may be prepared by mixing at least one excipient, for example, starch, calcium carbonate, sucrose or lactose, gelatin, and the like with one or more compounds.

Furthermore, in addition to simple excipients, lubricants such as magnesium stearate and talc may also be used. A liquid formulation for oral administration corresponds to a suspension, an emulsion, a syrup, and the like, and the liquid formulation may include, in addition to water and liquid paraffin which are simple commonly used diluents, various excipients, for example, a wetting agent, a sweetener, an aromatic, a preservative, and the like. A formulation for parenteral administration may include an aqueous sterile solution, a non-aqueous solvent, a suspension, an emulsion, a freeze-dried preparation, and a suppository. As the non-aqueous solvent and the suspension solvent, it is possible to use propylene glycol, polyethylene glycol, a vegetable oil such as olive oil, an injectable ester such as ethyl oleate, and the like. As a base of the suppository, it is possible to use Witepsol, Macrogol, Tween 61, cacao butter, laurin fat, glycerogelatin, and the like.

Further, the pharmaceutical composition of the present invention may have any one formulation selected from the group consisting of a tablet, a pill, a powder, a granule, a capsule, a suspension, an emulsion, a syrup, a sterilized aqueous solution, a non-aqueous solvent, a suspension, an emulsion, a freeze-dried preparation, and a suppository.

Meanwhile, as another aspect of the present invention, the present invention provides a method for preventing or treating neurodegenerative diseases, the method including administering the pharmaceutical composition to an individual.

As used herein, the term “individual” refers to all animals, including humans, who are likely to develop or have developed the neurodegenerative disease. By administering the compositions of the present invention to an individual, symptoms induced by a neurodegenerative disease may be alleviated or treated.

As used herein, the term “alleviation” refers to all actions that ameliorate or beneficially change symptoms of a neurodegenerative disease by administering the pharmaceutical composition according to the present invention.

The composition of the present invention is administered in a pharmaceutically effective dose.

As used herein, the term “administration” refers to introduction of the pharmaceutical composition of the present invention into an individual by any appropriate method, and the pharmaceutical composition of the present invention can be administrated through various routes, either oral or parenteral, that can reach the target tissues.

The pharmaceutical composition of the present invention may be appropriately administered to an individual according to the typical method, administration route and dosage used in the art, according to the purpose or need. Examples of the administration route include oral, parenteral, subcutaneous, intraperitoneal, intrapulmonary, and intranasal administration, and for parenteral injection includes intramuscular, intravenous, intraarterial, intraperitoneal, or subcutaneous administration.

In addition, an appropriate dosage and frequency of administration may be selected according to methods known in the art, and the dose and administration frequency of the pharmaceutical composition of the present invention actually administered can be appropriately determined by various factors such as the type of symptoms to be treated, administration route, sex, health status, diet, age and weight of the individual and severity of disease.

In the case of a viral vector, the administration frequency may be once or more per year, and when the viral vector is repeatedly administered, it may be administered at intervals of 1 day to 1 month, or 1 month to 1 year. Furthermore, when the pharmaceutical composition is a non-viral vector, it may be administered once or more a year, and when the non-viral vector is repeatedly administered, it may be administered at intervals of 12 to 24 hours and 1 to 14 days. Further, when the pharmaceutical composition is a protein, it may be administered once or more a year, and when it is repeatedly administered, it may be administered at intervals of 1 day to 1 month, or 1 month to 1 year.

As used herein, the term “pharmaceutically effective dose” refers to an dosage sufficient to treat a neurodegenerative disease at a reasonable benefit/risk ratio applicable to medical use, and an effective dosage level may be determined according to factors including the type of individual, the severity of disease, age, sex, the activity of drugs, sensitivity to drugs, administration time, administration route, excretion rate, treatment period, and simultaneously used drugs, and other factors well known in the medical field.

The pharmaceutical composition of the present invention may be administered as an individual therapeutic pharmaceutical agent or may be administered in combination with other therapeutic pharmaceutical agents, and may be administered sequentially or simultaneously with therapeutic pharmaceutical agents in the related art. Also, the composition of the present invention may be administered in a single dose or multiple doses. It is important to administer the composition in a minimum effective dose that can obtain the maximum effect without any side effects, in consideration of all the aforementioned factors, and this dose may be easily determined by those skilled in the art.

Further, the present invention provides a health functional food for preventing or ameliorating neurodegenerative diseases, containing at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same.

The “interleukin 10,” “glial cell line-derived neurotrophic factor,” “glutamate decarboxylase,” “protein,” “gene,” “neurodegenerative disease,” “prevention,” and the like may fall within the above-described ranges.

The term “amelioration” may mean all actions that at least reduce a parameter associated with the condition to be treated, for example, the severity of symptoms. In this case, in order to prevent or alleviate a neurodegenerative disease, the health functional food may be used simultaneously with or separately from a drug for treatment before or after the onset of the corresponding disease.

The health functional food may be used by adding an active ingredient as it is to food or may be used together with other foods or food ingredients, and may be appropriately used according to a typical method. The mixing amount of the active ingredient may be suitably determined depending on its purpose of use (for prevention or alleviation). In general, the health functional food may be added in an amount of about 15% by weight or less, more specifically about 10% by weight or less, based on the raw materials when manufacturing food or beverages. However, for long-term intake for the purpose of health and hygiene or for the purpose of health control, the amount may be below the above-mentioned range.

The health functional food further includes one or more of a carrier, a diluent, an excipient, and an additive, and thus, may be formulated into one selected from the group consisting of a tablet, a pill, a powder, a granule, a powder, a capsule, and a liquid formulation. Foods to which the compound according to an aspect can be added include various foods, powders, granules, tablets, capsules, syrups, beverages, gums, teas, vitamin complexes, health functional foods, and the like.

Specific examples of the carrier, excipient, diluent, and additive may be one or more selected from the group consisting of lactose, dextrose, sucrose, sorbitol, mannitol, erythritol, starch, acacia rubber, calcium phosphate, alginate, gelatin, calcium phosphate, calcium silicate, microcrystalline cellulose, polyvinylpyrrolidone, cellulose, polyvinylpyrrolidone, methyl cellulose, water, sugar syrup, methyl cellulose, methyl hydroxybenzoate, propyl hydroxybenzoate, talc, magnesium stearate, and mineral oil.

In addition to containing the active ingredient, the health functional food may contain other ingredients as essential ingredients without any particular limitation. For example, the health functional food may contain various flavoring agents or natural carbohydrates, and the like as an additional ingredient, as in a typical beverage. Examples of the above-described natural carbohydrates include typical sugars such as monosaccharides, for example, glucose, fructose and the like; disaccharides, for example, maltose, sucrose and the like; and polysaccharides, for example, dextrin, cyclodextrin and the like, and sugar alcohols such as xylitol, sorbitol, and erythritol. As the flavoring agent other than those mentioned above, a natural flavoring agent (thaumatin, a stevia extract (for example, rebaudioside A, glycyrrhizin and the like)), and a synthetic flavoring agent (saccharin, aspartame and the like) may be advantageously used. The proportion of the natural carbohydrate may be appropriately determined by the choice of a person skilled in the art.

The health functional food according to an aspect may contain various nutrients, vitamins, minerals (electrolytes), flavoring agents such as synthetic flavoring agents and natural flavoring agents, colorants and fillers (cheese, chocolate, and the like), pectic acid and salts thereof, alginic acid and salts thereof, organic acids, protective colloid thickeners, pH adjusting agents, stabilizers, preservatives, glycerin, alcohols, carbonating agents used in carbonated beverages, or the like, in addition to the additives mentioned above. Such components may be used independently or in combination, and the proportion of these additives may also be appropriately selected by those skilled in the art.

In addition, the present invention provides a method for preventing or treating neurodegenerative diseases, the method including administering at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same to an individual in need thereof.

The “interleukin 10,” “glial cell line-derived neurotrophic factor,” “glutamate decarboxylase,” “protein,” “gene,” “neurodegenerative disease,” “prevention,” “treatment,” “individual” and the like may fall within the above-described ranges.

Furthermore, the present invention provides a preparation of at least two selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same for preparing a preventive or therapeutic pharmaceutical agent for neurodegenerative diseases.

The “interleukin 10,” “glial cell line-derived neurotrophic factor,” “glutamate decarboxylase,” “protein,” “gene,” “neurodegenerative disease,” “prevention,” “treatment” and the like may fall within the above-described ranges.

Hereinafter, preferred examples for helping with understanding of the present invention will be suggested. However, the following examples are provided only so that the present invention may be more easily understood, and the content of the present invention is not limited by the following examples.

### EXAMPLES

### Example 1. Confirmation of Synergistic Effect of Co-Administration of hIL-10 and hGDNF on Amelioration of Neurodegenerative Disease

To conform the effect of rAAV5-hIL-10/hGDNF, one of the therapeutic pharmaceutical agents of the present invention on a neurodegenerative disease, saline, rAAV5-hIL-10, rAAV5-hGDNF and rAAV5-hIL-10/hGDNF were administrated to neurodegenerative disease animal model to observe the suppression of dopaminergic neuronal cell death and the improvement of motor function.

### 1-1. Preparation of Animal Model

It is known that motor malfunction is showed in both animal and human with neurodegenerative disease due to changes of neurotransmitter in brain. An animal model to observe the effect of the gene therapeutic pharmaceutical agent of the present invention on ameliorating neurodegenerative diseases was induced in C57BL/6 mouse. To produce a neurodegenerative disease mouse model, 6-hydroxydopamine (6-OHDA), which is known to induce dopaminergic neuronal cell death and abnormal excitatory neurotransmission in the subthalamic nucleus (STN), was injected into the left striatum (ST) of the mouse. A specific method for inducing a neurodegenerative disease mouse model is as follows.

The mice were anesthetized by intraperitoneal administration of an Alfaxan-sedator mixture, the head of the anesthetized animal was fixed using the nose clip and ear bars of a stereotaxic instrument (RWD Life Science), and then the height of the bregma and lambda of the mouse was measured to adjust the position such that the head was horizontal. After the coordinates of a desired brain target site were confirmed based on the bregma, a hole was made using a micromotor drill (Seashin) in the skull at the confirmed position. About 2 μL of test articles (may be saline, 0.1% ascorbic acid, 6-hydroxydopamine (OHDA), or rAAV5-GFP, and may vary depending on each experiment) were administrated by using a Hamilton syringe (Hamilton) (a 10 μL syringe was used, but is not limited thereto) at a rate of 0.5 μL/min with a stereotaxic injection pump (Dongbang Hitech Inc.). After injection, the brain skin was sutured using black silk, disinfected with povidone, returned to the their homecage, and then used an infrared heater to stabilize the body temperature until it woke up from anesthesia.

1-2. Confirmation of Protective Effect of Co-Administration of hIL-10 and hGDNF on Dopaminergic Neurons in Animal Model

Specifically, in order to confirm the effect of saline, rAAV5-hIL-10, rAAV5-hGDNF and rAAV5-hIL-10/hGDNF on the suppression of dopaminergic neuronal cell death, after pretreating the mice with saline, rAAV5-hIL-10, rAAV5-hGDNF and rAAV5-hIL-10/hGDNF was pretreated and 2 weeks later, neurodegenerative disease animal model was induced by injecting 6-OHDA in mouse. 3,3′-diaminobenzidine staining (DAB staining) was performed to confirm the protective effect on dopaminergic neurons in the striatum (ST) in which the axons of the substantia nigra pars compacta (SNpc) and SNpc neuronal cell body. In the experiment, 6 weeks after administration of saline, rAAV5-hIL-10, rAAV5-hGDNF and rAAV5-hIL-10/hGDNF (4 weeks after administration of 6-OHDA), the mice were sacrificed and harvested the brain, sectioned tissue was treated with 1% H₂O₂ for 15 minutes in order to prevent non-specific color development, and then to detect tyrosine hydroxylase (TH), which is known as a representative dopaminergic neuronal marker, the sectioned tissue was treated with an anti-TH antibody and incubated at a temperature of 4° C. for 8 hours or more. The tissue was treated with a biotinylated antibody to adhere to the anti-TH antibody, an avidin-biotin enzyme conjugate was bound to the biotinylated antibody using an ABC kit, and then the bound biotin was colored brown using a DAB kit and then observed under a microscope, in bright field.

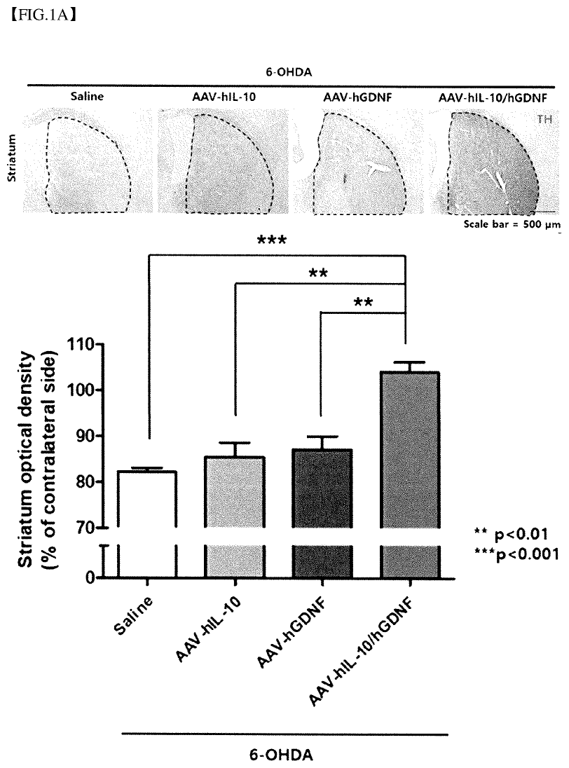

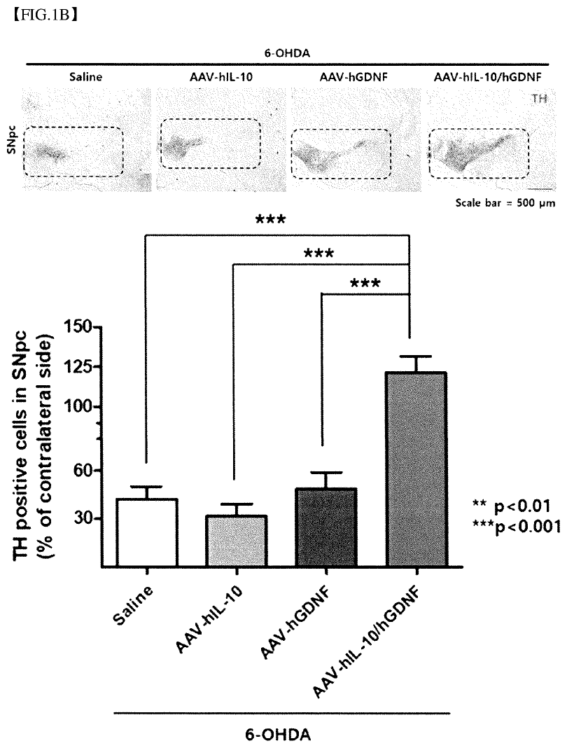

As a result, as shown in FIG. 1A, it could be confirmed that a group pretreated with rAAV5-hIL-10/hGDNF showed a significantly lower level of dopaminergic neuronal cell death in the striatum compared to groups pretreated with saline, rAAV5-hIL-10 or rAAV5-hGDNF alone, and further, even the case where a group was pretreated with rAAV5-hIL-10/hGDNF showed a significantly lower level of dopaminergic neuronal cell death in the SNpc compared to the case where a group was not pretreated with rAAV5-hIL-10/hGDNF (pretreated with saline, rAAV5-hIL-10, or rAAV5-hGDNF alone) (FIG. 1B).

1-3. Confirmation of Amelioration Effect of rAAV5-hIL-10/hGDNF on Hypokinesia Symptoms in Neurodegenerative Disease Animal Model

In addition, a rotation test was performed to confirm the motor function improvement effects of saline, rAAV5-hIL-10, rAAV5-hGDNF and rAAV5-hIL-10/hGDNF. For the rotation test, 0.4 mg/mL apomorphine was dissolved in saline, 4 mg/kg of apomorphine was subcutaneously administered to each neurodegenerative disease mouse model, and after 5 minutes, the direction and degree of movement of the animal were recorded using a camera for 20 minutes, and the recorded video was analyzed the amount of clockwise rotation of the animal.

As a result, the gene therapy (rAAV5-hIL-10/hGDNF) of the present invention showed an improvement in motor activity compared to saline, rAAV5-hIL-10 or rAAV5-hGDNF administrated mice, an effect of ameliorating a decline in motor function was shown (FIG. 1C).

1-4. Confirmation of Long-Tern Therapeutic Effect of rAAV5-hIL-10/hGDNF on Neurodegenerative Disease

In order to see the long-term therapeutic efficacy of rAAV5-hIL-10/hGNDF in persistent neurodegenerative disease, one of present invention, rAAV5-hIL-10/hGDNF, one of the present invention, was administered to 6-OHDA-induced animal models one week later. Specific experimental methods are shown in the following Table 1.

TABLE 1

| | | Injection materials | |
|---|---|---|---|
| | | 6-OHDA lesion | |
| Group | Animals | surgery | Test article injection |
| 0.1% AA + Saline | ICR, male | ST: 2 μL 0.1% AA | ST: 2 μL Saline |
| 6-OHDA + rAAV5-GFP | (8 weeks) | ST: 16 μg 6-OHDA/ | ST: rAAV5-GFP 1 × 10¹⁰ VG/2 μL |
| 6-OHDA + rAAV5- | | 2 μL 0.1% AA | ST: rAAV5-hIL-10/hGDNF 1 × 10¹⁰ |
| hIL-10/hGDNF | | | VG/2 μL |

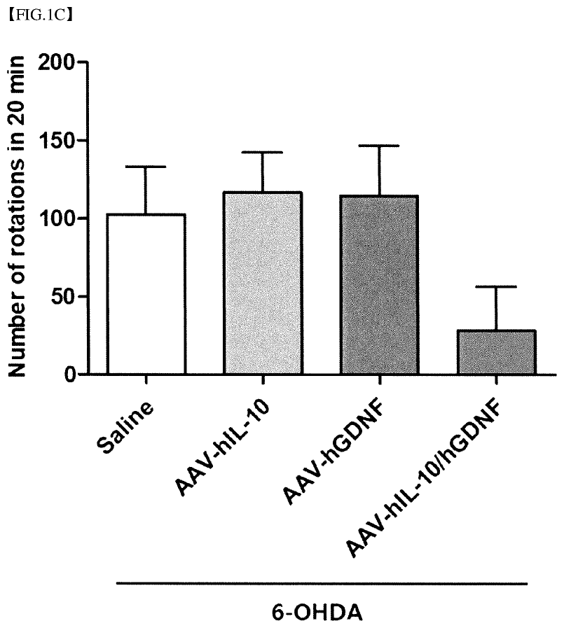

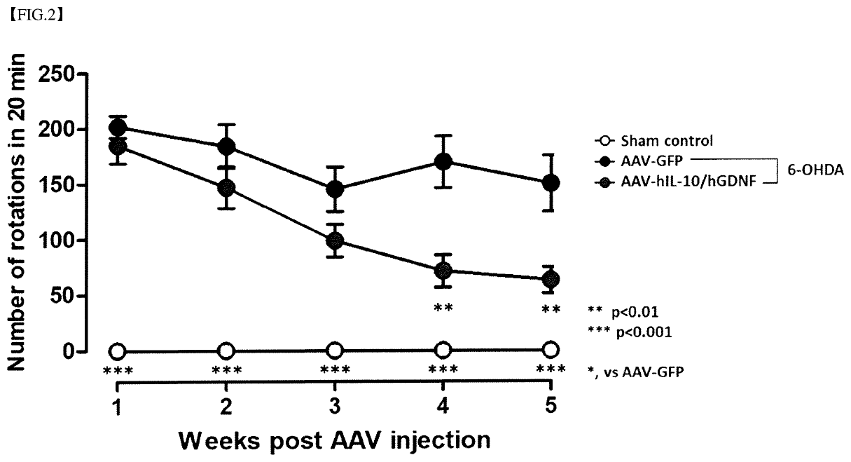

As a result of confirming the effect of ameliorating an abnormal behavior of a neurodegenerative disease when rAAV5-hGDNF/hIL-10 was post-treated and conducted a rotation test in the same manner as in Example 1-3 above, as shown in FIG. 2, it could be confirmed that even when rAAV5-hIL-10/hGDNF alone was administered, the abnormal behavior induced by 6-OHDA was ameliorated, and that such an effect was maintained for a long time even after 5 weeks had passed after the administration of rAAV5-hIL-10/hGDNF.

Through the results described above, the present inventors could confirm that when the gene therapeutic pharmaceutical agent (rAAV5-hIL-10/hGDNF) of the present invention is administered, it is possible to not only prevent but also treat the hypokinesia symptoms of a neurodegenerative disease and the preventive and therapeutic effects are maintained for a long period of time.

### Example 2. Confirmation of Neurodegenerative Disease Preventive Effect when rAAV5-hIL-10/hGDNF and rAAV5-hGAD65 are Simultaneously Administered

2-1. Confirmation of Protective Effects of rAAV5-hIL-10/hGDNF and rAAV5-hGAD65 on Dopaminergic Neurons in Animal Model

In order to confirm the effect of rAAV5-hIL-10/hGDNF and rAAV5-hGAD65 (simultaneously administered), which are one of the gene therapeutic pharmaceutical agents of the present invention, on the suppression of dopaminergic neuronal cell death, after pretreating the mice with rAAV5-hIL-10/hGDNF and rAAV5-hGAD65, 2 weeks later, a neurodegenerative disease was induced to mouse by administering 6-OHDA. The procedure was performed in the same manner as in Example 2-2 to confirm the protective effect on dopaminergic neurons in the striatum (ST) in which the axons of the substantia nigra pars compacta (SNpc) and SNpc neurons.

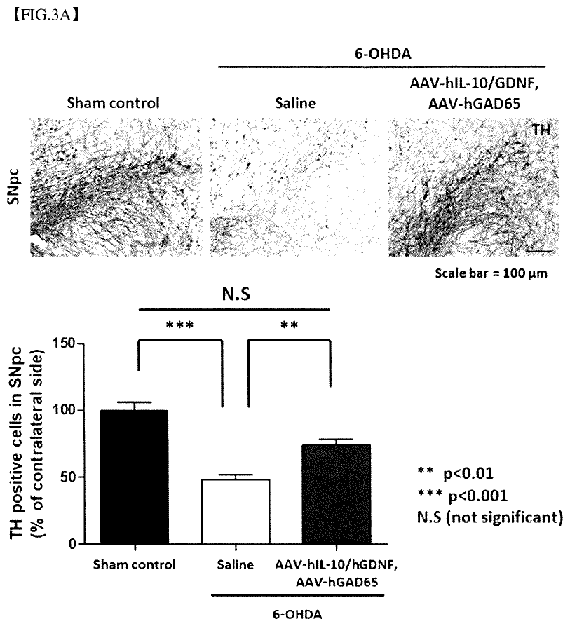

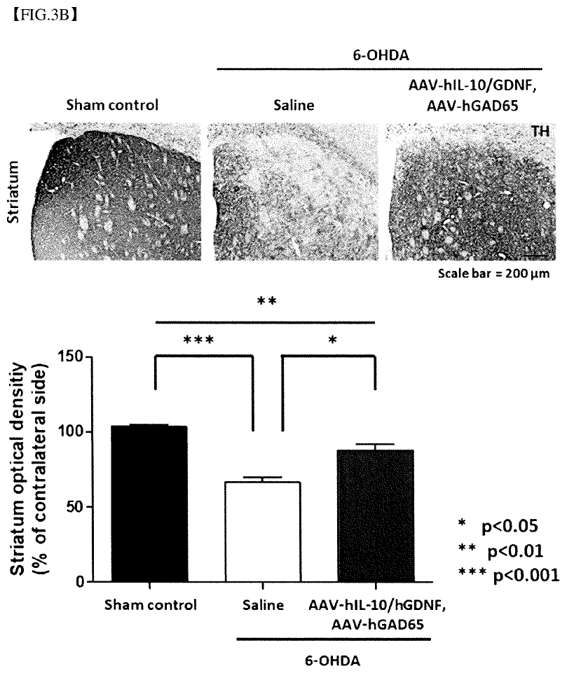

As a result, as shown in FIG. 3A, a group pretreated with rAAV5-hIL-10/hGDNF and rAAV5-hGAD65 showed a significantly lower level of dopaminergic neuronal cell death in the striatum compared to the no-pretreated with rAAV5-hIL-10/hGDNF and rAAV5-hGAD65 group, and, as shown in FIG. 3B, it could also be confirmed that the group pretreated with rAAV5-hIL-10/hGDNF and rAAV5-hGAD65 showed a significantly lower level of dopaminergic neuronal cell death in the SNpc compared to the group treated with 6-OHDA alone.

2-2. Confirmation of Amelioration Effects of rAAV5-hIL-10/hGDNF and rAAV5-hGAD65 on Hypokinesia Symptoms in Neurodegenerative Disease Animal Model

Two weeks before inducing disease animal mouse by the method of Example 2-1 above, the mice were pretreated with rAAV5-hIL-10/hGDNF and rAAV5-hGAD65, and a rotation test (apomorphine induced rotation) and a rota-rod test were performed on the neurodegenerative disease mouse model produced by the method described above. Specific experimental methods are shown in the following Table 2.

TABLE 2

| | | Injection materials | |
|---|---|---|---|
| | | | 6-OHDA lesion |
| Group | Animals | Test article | surgery |
| Saline + 0.1% AA | C57BL/6, male | SN: 2 μL Saline, | ST: 2 μL 0.1% |
| | (9 weeks) | ST: 2 μL Saline | ascorbic acid (AA) |
| Saline + 6-OHDA | | SN: 2 μL Saline, | ST: 18 μg 6-OHDA/ |
| | | ST: 2 μL Saline | 2 μL 0.1% AA |
| rAAV5-hIL-10/hGDNF + | | SN: rAAV5-hGAD65 | |
| rAAV5-hGAD65 + 6-OHDA | | 5 × 10¹⁰ VG/2 μL, | |
| | | ST: rAAV5-hGDNF/hIL- | |
| | | 10 5 × 10¹⁰ VG/2 μL | |

### \textcircled{1} Rotation Test Results

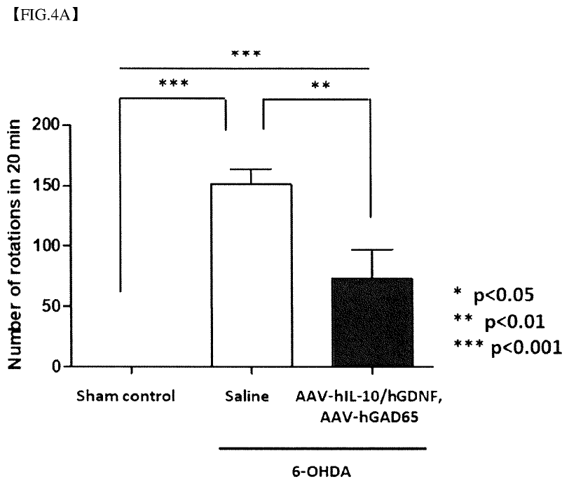

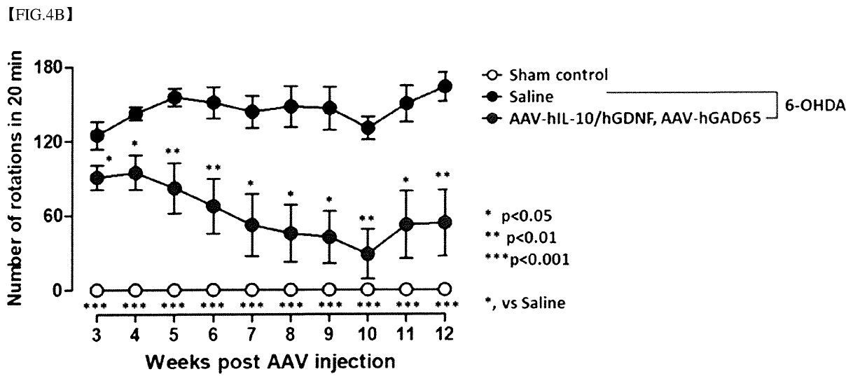

The rotation test was performed in the same manner as in Example 1-3. As a result, the neurodegenerative model preinjected with the gene therapy product of the present invention showed a significant improvement in motor function compared to the saline injection group after 4 weeks of 6-OHDA injection. (FIG. 4A). Also, as shown in FIG. 4B, it was found that there was a significant therapeutic effect on hypokinesia in 6-OHDA induced neurodegenerative model with pretreatment of therapeutic gene therapy compared to the saline injected group even after 10 weeks of neurodegenerative modeling. (FIG. 4B).

Through the results described above, the present inventors could confirm that when the gene therapeutic pharmaceutical agent (simultaneous administration of rAAV5-hIL-10/hGDNF and rAAV5-hGAD65) of the present invention is administered, it is possible to prevent the hypokinesia symptoms of the neurodegenerative disease and the preventive effect is maintained for a long period of time.

### \textcircled{2} Rota-Rod Test Results

In the rota-rod test, the mice were placed on a rota-rod behavioral test apparatus cylinder and then were given time to adapt to the apparatus by rotating the disk clockwise at a speed of 5 RPM for 2 minutes. The time the mouse walked on the cylinder was measured while rotating the cylinder at various RPMs, the experiment was performed in triplicate per individual, and the average of the three measurement values was shown as the representative value of the individual.

As a result, as shown in FIG. 4C, pre-treatment of rAAV5-hIL-10/hGDNF and rAAV5-hGAD65, one of the present inventions, showed motor improvement at a level similar to that of sham control groups which only 0.1% ascorbic acid administrated in 2 weeks, at which time point the 6-OHDA injection significantly reduced motor function.

### Example 3. Confirmation of Effect of Simultaneous Administration of hIL-10 and hGDNF Supernatant or hIL-10, hGDNF, and hGAD65 Supernatant Produced by Each Therapeutic Gene Plasmid Transfection on Suppression of Neuronal Cell Death Caused by Oxidative Stress

It was aimed to see the anti-apoptotic effect of neuronal cell caused by oxidative stress in co-administration of hIL-10 and hGDNF supernatant or simultaneous administration of hIL-10, hGDNF, and hGAD65 supernatant. The combination supernatant was produced by each therapeutic gene plasmid transfection. Cell viability was observed after inducing neuronal cell death by applying H2O2 or 6-OHDA, and hIL-10 and hGDNF supernatant or simultaneous administration of hIL-10, hGDNF, and hGAD65 supernatant produced by each therapeutic gene plasmid transfection was pre-treated.

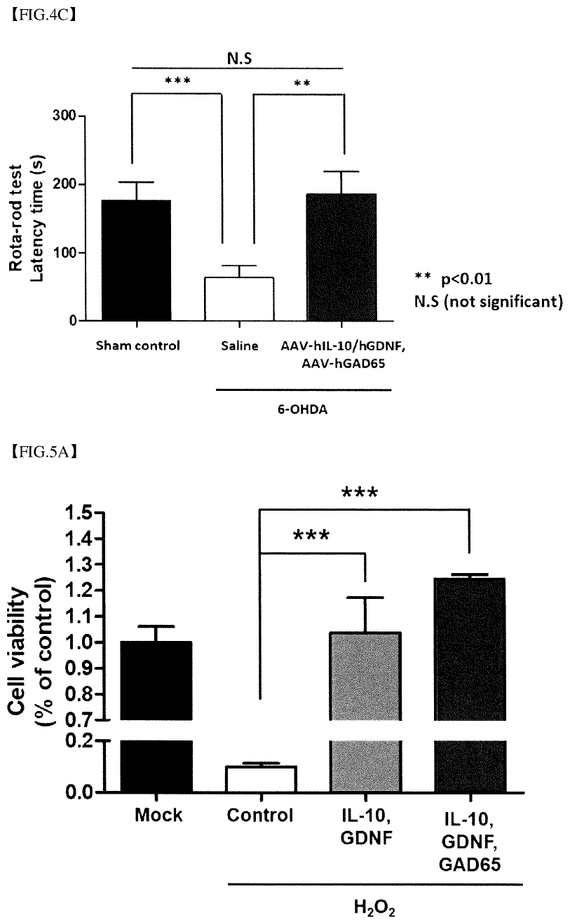

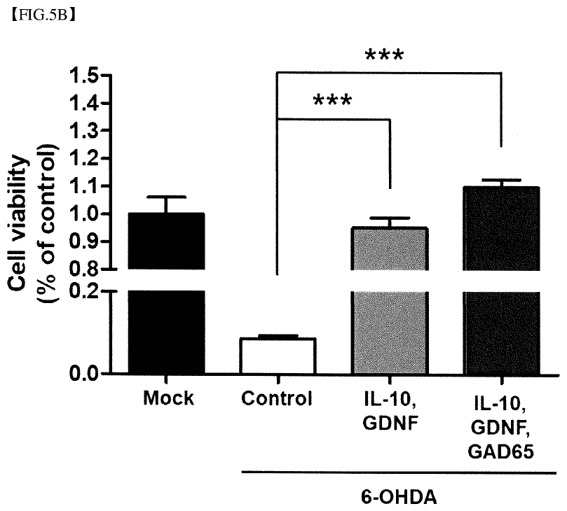

As a result, as can be seen in FIGS. 5A and 5B, it was confirmed that both co-administration of hIL-10 and hGDNF supernatant produced by each therapeutic gene plasmid transfection or simultaneous administration of hIL-10, hGDNF and hGAD65 supernatant produced by each therapeutic gene plasmid transfection suppressed neuronal cell death.

Through these results, it could be seen that not only the genes of hIL-10 and hGDNF, but also their respective proteins have preventive or therapeutic effects on neurodegenerative diseases, and that they are effective against various neurodegenerative diseases including Parkinson's disease because they have a neuroprotective effect regardless of the cause of neuronal cell death.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

1. A pharmaceutical composition for preventing or treating a degenerative brain disease, comprising two or more selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same.

2. The pharmaceutical composition of claim 1, comprising the IL-10 protein or the gene encoding the same and the GDNF protein or the gene encoding the same.

3. The pharmaceutical composition of claim 2, further comprising the GAD protein or the gene encoding the same.

4. The pharmaceutical composition of claim 1, wherein the gene is comprised in a vector.

5. The pharmaceutical composition of claim 4, wherein the vector is a viral or non-viral vector.

6. The pharmaceutical composition of claim 5, wherein the viral vector is one or more selected from the group consisting of adenoviruses, adeno-associated viruses (AAV), herpes simplex virus, lentiviruses, retroviruses, cytomegalovirus, baculoviruses, and poxviruses.

7. The pharmaceutical composition of claim 5, wherein the non-viral vector is one or more selected from the group consisting of plasmids, liposomes, cationic polymers, micelles, emulsions, and solid lipid nanoparticles.

8. The pharmaceutical composition of claim 1, wherein the IL-10 protein is an amino acid sequence represented by SEQ ID NO: 1.

9. The pharmaceutical composition of claim 1, wherein the IL-10 encoding gene is a base sequence represented by SEQ ID NO: 2 or SEQ ID NO: 3.

10. The pharmaceutical composition of claim 1, wherein the GDNF protein is an amino acid sequence represented by SEQ ID NO: 4.

11. The pharmaceutical composition of claim 1, wherein the GDNF protein encoding gene is a base sequence represented by SEQ ID NO: 5 or SEQ ID NO: 6.

12. The pharmaceutical composition of claim 1, wherein the GAD is GAD 65 or GAD 67.

13. The pharmaceutical composition of claim 1, wherein the GAD protein is an amino acid sequence represented by SEQ ID NO: 7 or SEQ ID NO: 9.

14. The pharmaceutical composition of claim 1, wherein the GAD protein encoding gene is a base sequence represented by SEQ ID NO: 8 or SEQ ID NO: 10.

15. The pharmaceutical composition of claim 1, wherein the degenerative brain disease is one or more selected from the group consisting of Parkinson's disease, dementia, Alzheimer's disease, Huntington's disease, frontotemporal lobar degeneration, dementia with Lewy bodies, vascular dementia, corticobasal degeneration, multiple system atrophy, progressive supranuclear palsy, primary lateral sclerosis, spinal muscular atrophy, stroke, cerebral infarction, head trauma, spinal cord injury, cerebral arteriosclerosis, Lou Gehrig's disease, and multiple sclerosis disease.

16. The pharmaceutical composition of claim 1, further comprising a physiologically acceptable carrier.

17. The pharmaceutical composition of claim 1, wherein the pharmaceutical composition is an injection.

18. The pharmaceutical composition of claim 1, wherein the pharmaceutical composition has an effect of ameliorating motor abnormalities caused by neuronal cell death.

19. The pharmaceutical composition of claim 18, wherein the neurons are motor neurons, sensory neurons or interneurons.

20. The pharmaceutical composition of claim 1, wherein the pharmaceutical composition has an effect of improving cognitive ability and memory.

21. A health functional food for preventing or ameliorating a degenerative brain disease, comprising two or more selected from the group consisting of an interleukin (IL-10) protein or a gene encoding the same, a glial cell line-derived neurotrophic factor (GDNF) protein or a gene encoding the same, and a glutamate decarboxylase (GAD) protein or a gene encoding the same.

22. A method for preventing or treating a degenerative brain disease, the method comprising administering two or more selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same to an individual in need thereof.

23. A use of two or more selected from the group consisting of an interleukin 10 (IL-10) protein or a gene encoding the same, a glial cell line-derived neurotrophic factor (GDNF) protein or a gene encoding the same, and a glutamate decarboxylase (GAD) protein or a gene encoding the same for preparing a preventive or therapeutic agent for degenerative brain diseases.
